(19) United States
(12) Patent Application Publication
(10) Pub. No.: US 2011/0159040 A1
(43) Pub. Date: Jun. 30, 2011

(54) NOVEL IMMUNOGENS AND METHODS FOR DISCOVERY AND SCREENING THEREOF

(75) Inventors: Richard Malley, Beverly, MA (US); Yingjie Lu, Chestnut Hill, MA (US); Kristin L. Moffitt, Woburn, MA (US)

(73) Assignee: CHILDREN'S MEDICAL CENTER CORPORATION, Boston, MA (US)

(21) Appl. No.: 12/981,706

(22) Filed: Dec. 30, 2010

Related U.S. Application Data
(60) Provisional application No. 61/290,914, filed on Dec. 30, 2009.
(60) Provisional application No. 61/313,450, filed on Mar. 12, 2010.

(51) Int. Cl. A61K 39/09; C07K 1/14; C07H 1/06; C07C 51/42; G01N 33/68; C12Q 1/02; A61P 37/04; A61P 31/04

(52) U.S. Cl. 424/244.1; 530/412; 536/25.4; 554/206; 536/127; 436/501; 435/29

(57) ABSTRACT

An aspect of the present invention provides for methods for identifying novel immunogens that when administered as vaccines elicit a cellular or humoral immunogenic response. In a particular embodiment, a method identified pneumococcal T-cell immunogens that elicit systemic IL-17A responses, and reduce or protect against pneumococcal colonization.

### CROSS REFERENCE TO RELATED APPLICATIONS

This application claims the benefit of priority under 35 U.S.C. §119(e) of U.S. Provisional Application No. 61/290,914 filed Dec. 30, 2009 and U.S. Provisional Application No. 61/313,450 filed Mar. 12, 2010, the contents of each of which are incorporated herein by reference in their entirety.

### BACKGROUND

Almost one million children in the developing world die of pneumococcal infections each year. Despite the effectiveness of the conjugate pneumococcal vaccines, problems with this approach remain including the expense of production and delivery, and resulting serotype replacement as demonstrated in several clinical trials and epidemiologic studies. One positive effect of the current capsular-based vaccines has been evident in the patient population that is not being vaccinated: herd immunity plays an impressive role in the current vaccine strategy. For each case of pneumococcal disease prevented in children, about three cases of pneumococcal disease are prevented in adults by herd immunity. In this context at least, prevention of pneumococcal colonization is a main goal of protein-based vaccine approaches, because blocking colonization will block disease.

Alternative pneumococcal vaccines that elicit serotype-independent immunity, and that may be more readily available to economically emerging countries are needed urgently. New antigens that can address this need would be very attractive. Additionally, current methods to identify immunogens focus on techniques that do not fully optimize the extraction or identification of the full antigen repertoire. This is true in the case of pneumococcus as well as other pathogens. A method that can identify a new set of antigens has potential to be impactful for the development of vaccines for a wide set of pathogens, including pneumococcus.

### SUMMARY

An aspect of the present invention provides for methods for identifying novel immunogens that when administered as vaccines elicit a cellular or humoral immunogenic response. In a particular embodiment, a method identified pneumococcal T-cell immunogens that elicit systemic IL-17A responses, and reduce or protect against pneumococcal colonization.

In one embodiment, protective immunogens are identified by killing an organism with an organic solvent. The organic solvent is removed, and the remaining materials re-hydrated in aqueous solution. This process releases various antigens in the liquid phase, which can then be harvested by centrifugation and collection of supernatants.

In another embodiment, the liquid phase is further size-fractionated, or separated by preparative SDS gel or other methods, following which individual fractions are evaluated for immune stimulation. The most promising fractions are then evaluated further to identify components. Component proteins can then be evaluated in combination or singly to determine which are the most immunogenic and protective.

Accordingly, the present approach identified pneumococcal T-cell immunogens that both induce a Th17-cell response and protect mice from colonization. These proteins, including SP0862, SP1534 and SP2070, show promise as vaccine candidates against colonization and sepsis. In a prefered embodiment, the novel pneumococcal immunogens are administered by mucosal immunization with an adjuvant, and reduce subsequent pneumococcal nasal colonization.

### DESCRIPTION OF THE FIGURES

FIG. 1 shows data from a stimulation of splenocytes with elutions from preparative SDS gel separation. The supernatant fraction (WCC sup) contains about 15% of total protein of whole cell vaccine killed by chloroform (WCC). Proteins in WCC sup were separated in a 4%-12% SDS gel and then eluted into fractions according to their mobility in the gel by a preparative SDS gel elution apparatus. Splenocytes from WCC immunized mice were stimulated with the same amount of protein from fraction 3 to 11 and their IL-17A production was measured by ELISA 3 days after stimulation.

FIG. 2 shows gels from the purification of individual proteins from E. coli. Proteins were cloned into competent E. coli cells using the pQE-30 plasmid; transformants were verified by sequencing. Proteins were expressed in successful transformants and pelleted. After lysing by sonication, his-tagged proteins in the cell lysis supernatant were purified over an agarose-Ni column. Eluted proteins were then desalted over a PD10 column and again purified by size exclusion gel filtration. Representative proteins are depicted on Coomassie stained SDS-PAGE gels.

FIG. 3 presents data from the stimulation of IL-17A production by purified proteins. Twelve proteins were selected from all of the proteins identified by Mass spectroscopy (Supplemental table I), cloned into and purified from E. coli. Splenocytes from WCC immunized mice (n=10) were stimulated with 10 μg/ml of each protein and IL-17A production was measured 6 days after stimulation. Bars represent median values for each stimulus. Each animal's IL-17A response to stimulation with DMEM media was considered background, and was therefore subtracted from the IL-17A values from protein stimuli.

FIG. 4 presents data on protection against colonization by intranasal immunization with a mixture of proteins. A mixture of 4 μg/ml of each SPN0435, SPN1534 and SPN2070 (CHB mix) was used to immunize mice twice one week apart with 1 μg of cholera toxin (CT) as adjuvant. Mice immunized with CT alone or a whole cell pneumococcal preparation with CT (WCB) constituted negative and positive controls, respectively. Blood was taken 3 weeks after second immunization. IL-17A production in vitro was determined in the blood samples incubated 6 days with pneumococcal whole-cell antigen (4A). The mice were challenged intranasally with serotype 6B strain 0603 four weeks post-immunization, and the density of pneumococcal colonization was determined 7 days later by plating dilutions of nasal washes (4B).

FIG. 5 demonstrates protection against colonization by individual proteins in C57B1/6 mice. Immunization and challenge schedule was the same as in FIG. 4. NP colonization density was compared by the Mann-Whitney U test or by the Kruskal-Wallis test with Dunn's correction for multiple comparisons using PRISM. *, p<0.05; **, p<0.01.

FIG. 6 demonstrates protection against colonization by individual proteins in outbred CD1 mice. Immunization and challenge schedule was the same as in FIG. 4. NP colonization density was compared by the Mann-Whitney U test or by the Kruskal-Wallis test with Dunn's correction for multiple comparisons using PRISM. *, p<0.05; **, p<0.01.

FIG. 7 shows data from stimulation of exposed mice with individual SP proteins.

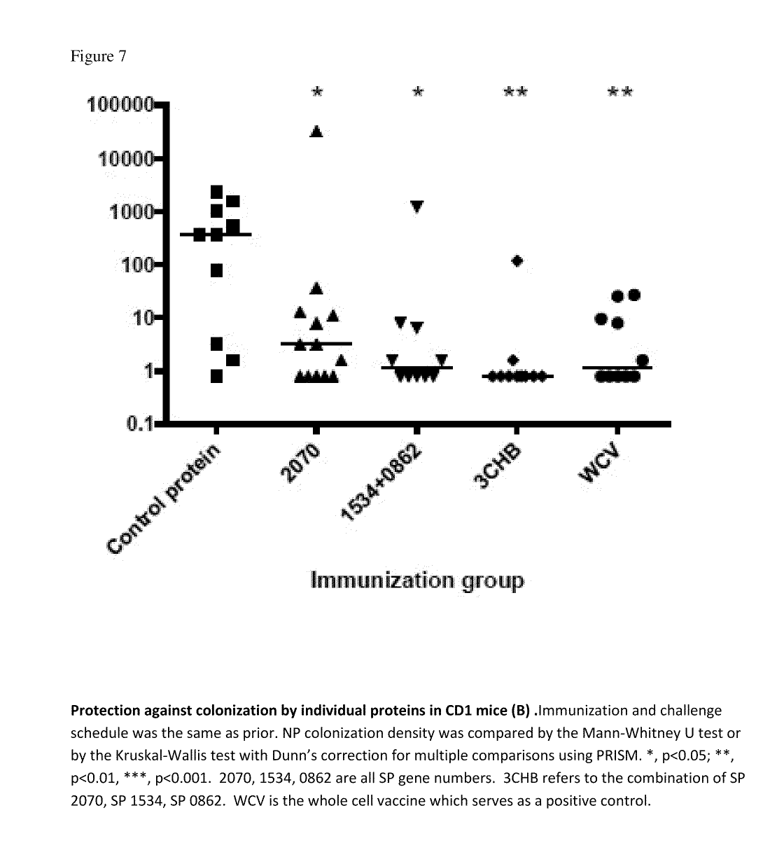

### DETAILED DESCRIPTION

### Antigen Discovery Method

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Two approaches proposed thus far to expand pneumococcal vaccination are based on protein subunit vaccines composed of purified pneumococcal antigens and/or on killed whole cell vaccines such as a whole cell vaccine (WCV) candidate.

Work on the pneumococcal whole cell vaccine candidate (WCV) has included transition towards GMP grade production. During this process, organic solvents were explored as an alternative to ethanol killing. It was discovered, surprisingly, that WCV killed with chloroform (WCC) was 100 1000× more potent than the ethanol-killed WCV. Additionally, when lyophilized WCC was reconstituted and spun down, the supernatant alone was highly protective in an animal colonization model. This lead to the hypothesis that the use of chloroform, which simply sublimates away during freezing and lyophilization, maintained soluble protective proteins of the WCV that were otherwise washed away after ethanol killing. The proteins in the WCC supernatant that contributed to its immunogenicity and protective capacities were then further characterized.

Thus, the present invention provides for methods for identifying antigenic candidates for vaccines. First, possible protective antigens are identified by killing a pathogen (e.g., an infectious bacteria such as pneumococcus or an infectious virus such as influenza) with one or more organic solvents. Suitable solvents include, among others, common solvents used in biological purification procedures, such as chloroform or trichloroethylene, TCE). The organic solvent or solvents can be removed by methods known in the art to evaporate or sublime organic fractions in mixtures, e.g., by lyophilyzation. The remaining materials are re-hydrated by adding water or other suitable aqueous solution. This process releases various antigens in the liquid phase, which can then be harvested by any methods common in the art, for example, by centrifugation or other phase separation techniques, and collection of supernatants. Additionally, various antigens that are not released in the liquid phase may be extracted by treatment of the remaining centrifuged portion with extraction techniques such as application of organic solvents, acids or bases, re-precipitation techniques, physical homogenization and/or separation, further fraction dispersion techniques, or other methods known in the art to fractionate and isolate components of solid centrifuged masses.

This method has led to surprising and important results. In the case of pneumococci, the soluble fraction of the chloroform killed bacterial preparation was one-hundred-times more protective than preparation in which the soluble fraction was washed away. Without being bound by theory, killing with chloroform or a similar solvent may uncover protective antigens that have not been identified when more traditional killing techniques (e.g., heat, ethanol, formalin), are used. Importantly, this process may be superior to more traditional techniques due to the preservation of antigens in the working mixtures. For example, it is likely that some antigens are washed away during processes that require an early supernatant washing step such as ethanol killing. It is also probable that techniques based on temperature, such as heat killing, may induce denaturation or aggregation of some antigens. Further, chemicals such as formalin have undesirable properties such as crosslinking and aggregating biological macromolecules. The present novel method herein maintains fractions in common organic solvents, and thus may avoid some of these potential issues.

Importantly, this method is not based on the mode of immunogenicity of the molecules identified. Therefore, the method allows for the isolation and identification of any type of microbial biological molecule that might elicit an immune response, including proteins, carbohydrates, lipids, nucleic acids, or small molecules. Any of these types of macromolecules that are not removed with the original organic solvent have the potential to serve as novel antigens. This method is also agnostic to the normal organismal location of the molecule, and therefore allows for identification of antigens that are external (e.g. surface expressed, secreted, etc.), internal (e.g. cytoplasmic, organelle-associated, etc.), membrane bound, or capsular (associated with pathogen encapsulation layer).

Additionally, this method is not based on specific pathogen characteristics, and therefore has broad applicability to various pathogens. For example, the present method may be used to identify novel antigens from bacteria, viruses, fungi, and parasites. Non-limiting examples of the antigen discovery method of the present invention includes identifying antigens from pathogenic bacteria and viruses including Staphylococci (including MRSA), Streptococci species (including Group A and B), Brucella, Enterococci species; Listeria, Bacillus (including anthrax), Corynebacteria, Neisseria meningitidis, Neisseria gonorrheae, Moraxella, typeable or nontypeablc Haemophilus, Haemophilus nontypeable, Pseudomonas aeruginosa and others, Salmonella typhi, non-typhi Salmonella, Shigella, Enterobacter, Citrobacter, Klebsiella, E. coli, Clostridia, Bacteroides, Chlamydiaceae, Mycoplasma, Legionella, Treponemes, Borrelia, Candida or other yeast or other fungi, Plasmodium, Amoeba, herpes viruses, cytomegalovirus, Epstein-barr virus, varicella-zoster virus, influenza, adenoviruses, enteroviruses, or hemorrhagic viruses.

This present unique method has identified novel T-cell antigens to pneumococci. Thus, the method of the invention may be particularly useful for antigen discovery in pathogens that require T-cell in addition to B-cell response. Therefore, pathogen targets of the present invention include those known or discovered to require T-cell or more specifically Th17 cell activity, including S. aureus, C. trichomatis, M. tuberculosis, viruses such as Herpes simplex virus, and others.

In order to confirm immunogenicity of the novel fractions, the liquid fraction can then be size-fractionated or separated by preparative SDS gel or other methods, following which individual fractions are evaluated for immune stimulation in a variety of assays. Example assays include those to directly measure antibody or T-cell responses, such as ELISA assays, cell sorting procedures, neutralization assays, or others known in the art. Additionally, it is potentially useful to monitor production of markers or secretions of cell-types, such as cytokines. Example assays include T cell assays, such as elicitation of IL-17A from immune animals, or the monitoring of other cytokines such as IFN-gamma, IL-4, etc., that can identify those fractions to which antibodies from immune animals bind strongly. The most-promising fractions may then be evaluated further to identify components, e.g., by mass spectroscopy or other techniques. Proteins can then be evaluated singly to determine which are immunogenic and protective. Since the separation isolation method of invention can be flexibly coupled to the immune system endpoints above, the method of the invention is useful to identify antigens that can be immunogenic in a variety of ways, including T-cell effector subtypes, antibody responses, or other adaptive or innate immune mechanisms.

The unique method has identified novel pneumococcal antigens, and demonstrates the utility of the approach to uncover novel immunogens from well-studied pathogens.

### Novel Antigens

One mechanism of protection against pneumococcal colonization has been elucidated with a WCV candidate that confers protection against both colonization and invasive disease in mice. (Malley et al., 69 Infect. Immun. 4870-73 (2001); Malley et al., 74 Infect. Immun. 4290-92 (2004).). Protection against colonization following immunization with WCV is antibody-independent and dependent on CD4+ T cells (Malley et al., 102 P.N.A.S. USA 102,4848-53 (2005); Trzcinski et al., 73 Infect. Immun. 7043-46 (2005)). The effector T cell is the CD4+ TH17 cell: neutralization of IL-17A with anti IL-17A antibodies diminishes protection by the WCV and ll-17A receptor knockout mice are not protected by the WCV. In contrast, IFN-gamma or IL-4 deficient mice (which are skewed away from THI or TH2 responses, respectively) are fully protected (Lu et al., 4 PLoS Pathogens. e1000159 (2008)). Rats and mice immunized with the WCV are also significantly protected against pneumococcal sepsis in two pneumonia models (Malley et al., 2001).

This unique, sequential method described above has herein identified novel pneumococcal T-cell antigens. These antigens, administered as mucosal vaccines with a cholera toxin adjuvant, elicit systemic IL17A and reduce or protect against intranasal pneumococcal colonization. More specifically, SPN2070 was completely protective. Both SPN0862 and SPN1534, although not fully protective, significantly reduced colonization.

It is feasible that a pneumococcal protein subunit vaccine would contain several antigens and/or be formulated with different or novel adjuvants, or incorporated in vaccine scaffolds, such as a fusion-conjugate (e.g., a fusion with a pneumolysoid and conjugation to a polysaccharide as proposed in Lu et al, Infection and Immunity, 2009) to improve immunogenicity and facilitate different routes of administration.

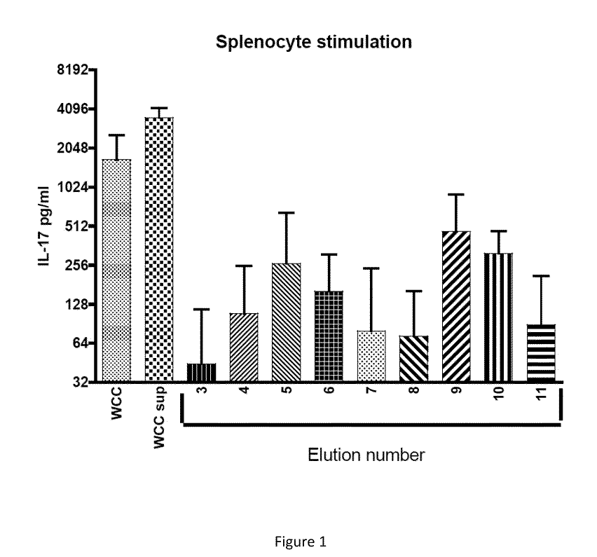

Several eluates with robust stimulatory potential were identified from the method applied to pneumococcus, yielding WCC. Of the fourteen eluates collected representing separation of the WCC supernatant, the nine eluates containing clear protein bands were used as stimuli. Several eluates clearly emerged as having higher potential to elicit Th17 cell activation. Data from these stimulations are depicted in FIG. 1. The predominant band(s) of the most stimulatory eluates (such as 9 and 10) were submitted for mass spectroscopy analysis.

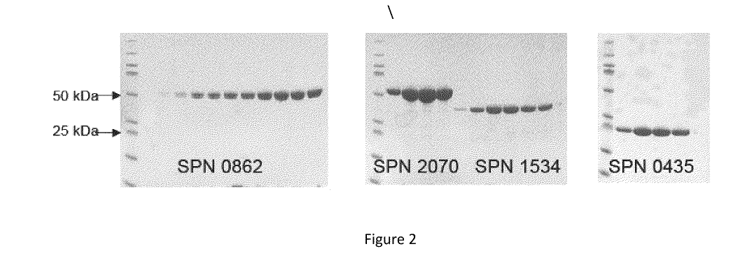

Mass spectroscopy identified multiple proteins within each band (range 13-23), but with some overlap in adjacent eluates. A compilation of data from mass spectroscopy analysis was used to generate a table of over forty proteins that were contained within the stimulatory WCC supernatant eluates. (See supplemental Table 1). Based on clinical safety criteria such as lack of human homology and conservation across all twenty-two sequenced pneumococcal strains, this panel was narrowed to twelve proteins. These proteins were then expressed and purified in an E. coli expression system. Protein gels of the purified proteins yielded single bands (FIG. 2) suggesting successful purification.

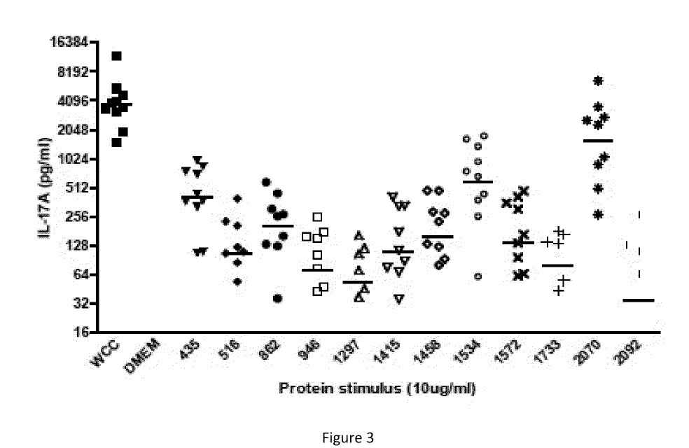

Immunogenic purified proteins were next identified by determining which purified proteins elicited the highest IL-17A response from splenocytes of WCC immunized mice, thus prioritizing the antigens that would move into animal immunization models. As shown in FIG. 3, several proteins met this criterion (SPN2070, SPN1534, SPN0435 and SPN0862). Though not meeting the self-imposed criterion, and thus not tested for protection, SPN516, SPN862, SPN946, SPN1297, SPN1415, SPN1458, SPN1572, and SPN1733 were also identified as novel pneumococcal antigens able to elicit immunogenic responses. Additionally, theprotein SPN2092 showed minimal stimulatory potential; as a protein expressed and purified by the same process, this protein would become a representative negative control in an immunization model.

This animal model revealed that a combination of proteins protects against colonization.

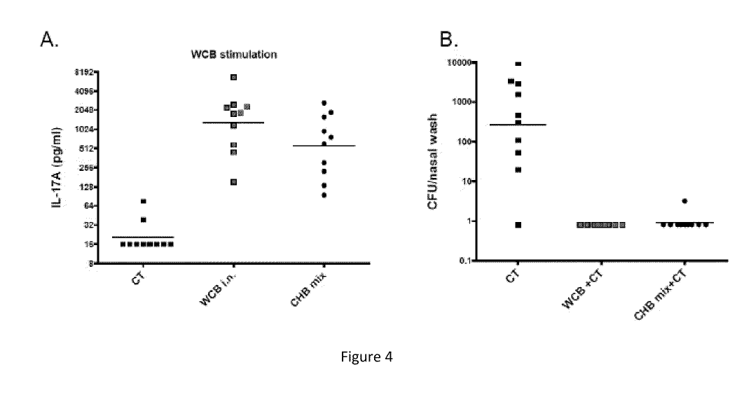

Initially, candidate proteins were evaluated using a combination vaccine containing the three antigens that were most stimulatory in the splenocyte stimulations (FIG. 3). The immunogenicity of this mixture containing SPN 0435, SPN 1534, and SPN 2070, when the whole blood of immunized animals was stimulated with the whole cell antigen, was robust (FIG. 4A). Extensive experience with WCV studies have indicated that post-immunization IL-17A values of >250 μg /ml in whole blood stimulated with whole cell antigen correlate well with protection from colonization (Lu et al., 2008). Animals immunized with the combination vaccine indeed were completely protected from colonization, and the combination vaccine was as protective as the WCV (FIG. 4B).

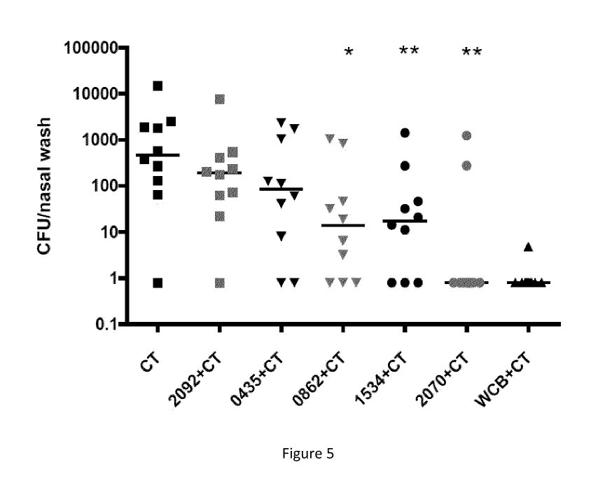

Individual purified protein also provided protection in a colonization model. Having shown that a combination of these proteins protected animals from colonization, which individual proteins contributed most to this protective capacity was explored. Using each of the proteins contained within the combination, plus an additional protein that was stimulatory in the splenocyte assay (SPN0862), animals were immunized with vaccines comprised of single proteins with CT. As shown in FIG. 5, SPN2070 was highly protective, essentially as protective as WCV. Additionally, SPN1534 and SPN0862 conferred statistically significant reduction in colonization compared with cholera toxin-immunized controls. Animals immunized with SPN2092 were not protected, validating the methods used to identify and predict those proteins, chosen from a larger pool of proteins that would be immunogenic and protective.

The novel antigens described here, namely SPN2070, SPN1534, and SPN0862, and SPN0435 demonstrate the utility of the method and provide new vaccine candidates. It has been suggested that SPN2070 might be used as a vaccine antigen, however it was previously unknown that this protein could elicit a T-cell specific response. Additionally, the remaining proteins have, to our knowledge, never before been described as possible antigens for a pneumococcal vaccine approach. Thus, the method presented here has approached a well-studied pathogenic bacteria and been able to identify proteins previously unknown to elicit immune-cell-specific antigenic response.

In some cases, it there may beadvantages to designing vaccines based on antigens that are “surface-expressed” rather than cytoplasmic. Annotated genomes often describes protein location based on homology with other identified proteins, but this may be an imperfect or often incorrect approach, as homologous proteins form two different organisms may not necessarily be located at the same site (nor have the same function) in both. Thus, an additional tool for determination of the location of the protein (surface versus other) may be very helpful and may also be used in the chloroform method described herein.

An embodiment of the present method comprises identifying a protein, “X” of interest, then removing the gene encoding for X from the organism, then replacing that gene with a gene encoding for a tagged version of the X protein (e.g., tagged with His, HA, OVA peptide, among others), which can be detected readily with monoclonal or polyclonal antibodies. After confirmation of the genetic construct, the organism is then grown, stained with an antibody that recognizes the tag (and is also fused to a fluorophore). Flow cytometry is then used to evaluate whether the antibodies are attached to the surface of the organism, in which case, the antigen can be deduced to be surface-expressed. Similar strategies using antibodies attached to magnetic beads can be used as well. For pneumococcus, for example, the organism can be evaluated in its encapsulated or unencapsulated form. An antigen can be surface expressed, but hidden under the capsule, for selection of antigen purposes, it may be advantageous to select an antigen that is both surface expressed and accessible despite capsulation.

The identified immunogenic proteins or mixtures thereof may be used in a multivalent or individual vaccine, which can be administered in many forms (intramuscularly, subcutaneously, mucosally, transdermally). For example, combinations or permutations of the twelve pneumococcal immunogens may be more efficacious against colonization versus disease. A combination of several immunogens with both characteristics may provide a superior vaccine.

Immunogenic compositions may contain adjuvants. As shown herein, cholera toxin was used as an adjuvant for intranasal administration, resulting in protection from pneumococcal colonization. Alum is an affective adjuvant for subcutaneous injection.

Alternatively, such proteins or mixtures may be useful in diagnostics.

Ongoing studies assess immunogenicity in outbred animal strains, and characterize the efficacy of these proteins in protecting against invasive disease in aspiration/sepsis models.

### EXAMPLES

### Example 1

### Pneumococcal Immunogens

### Protein Isolation

Initial efforts to isolate these proteins via gel filtration of the WCC supernatant did not yield discrete enough separation of proteins. Ultimately, preparative SDS gel transverse elution of the WCC supernatant improved separation of the proteins. WCC supernatant with SDS buffer was loaded into 10 wells of a precast 4%-12% Bis/Tris SDS gel with −100 μg protein/lane and run at 200V over 30 min in MES-SDS buffer. This gel was equilibrated in 2 mM phosphate buffer for 20 min fresh buffer three times to minimize SDS. The equilibrated gel was cut to size to fit the BioRad Mini Gel Eluter apparatus. Proteins were transversely eluted through the thickness of the gel with 90 mA of current for 20 mM. Eluates were collected into the elution chambers beneath the gel and harvested by vacuum apparatus. This method yielded fourteen eluates with one or two protein bands per eluate. The proteins within each eluate were visualized on silver stained SDS gel; bands within each eluate were reproducible from elution to elution; eluates were combined for further use.

### Protein Identification

Eluates were used as stimuli on splenocytes from C57B1/6 mice immunized with WCC to determine which eluates contained proteins capable of eliciting IL-17A production. Mice (n=10) were immunized intranasally with WCC one week apart. Three weeks following their second immunization, spleens were harvested and processed into a cell suspension in DMEM with L-glutamine/10% FCS/2ME/cipro. The protein concentration within each eluate was determined by quantitative BCA assay; eluates were used as stimuli with each stimulus normalized to the lowest concentration among the eluates. Supernatants were harvested after six days and assayed for IL-17A by ELISA (R&D Biosciences). We then submitted the predominant band or bands from the most stimulatory eluates for mass spectroscopic analysis.

### Protein Expression and Purification

Antigens were selected from the compiled mass spectroscopy data based on clinical safety criteria such as lack of human homology and conservation across sequenced pneumococcal strains. Selected antigens (n=12) were cloned into competent E. coli cells for expression using the pQE-30 plasmid vector incorporating a 6x-histidine (his) tag. Transformed E. coli colonies were sequenced for the protein of interest; transformants containing successfully cloned proteins were grown and induced for protein expression using IPTG. After overnight incubation allowing expression, transformants were spun down and pellets were lysed by sonication. The proteins of interest were purified from the lysed cell supernatant over a column using agarose-Ni beads to bind the His-tag; proteins were eluted in imidazole buffer after careful washing of the column. Protein-containing elutions were further purified over a desalting column prior to use in cellular stimulation assays.

### Assessing Immunogenicity of Purified Proteins

In an effort to prioritize which of the twelve purified proteins for test vaccines in animal models, each protein's immunogenicity was assessed in the splenocyte stimulation assay performed similarly to the assay described above used to identify which eluates contained stimulatory proteins. Splenocytes from a separate cohort of ten WCC-immunized C57B1/6 animals were stimulated with 10 μg/ml of each of the twleve proteins.

### Assessing the Protective Capacity of Purified Proteins Against Colonization

In the first immunization experiment, C57B1/6 mice (n=10 per group) were immunized

intranasally twice one week apart with a combination of the three most stimulatory proteins (SPN0435, SPN1534, and SPN2070). The combination vaccine contained 4 μg of each protein per vaccine dose. Vaccines were prepared with cholera toxin (CT) adjuvant. Control cohorts were immunized with WCV and CT or CT alone. Three weeks following their 2nd immunization, animals were bled; whole blood was stimulated with the whole cell antigen and IL-17A was measured from the supernatant to assess immunogenicity. One week after being bled, animals were challenged intranasally with a live type 6B pneumococcal strain. One week after challenge, animals were sacrificed and nasal washes were obtained and cultured to assess density of colonization.

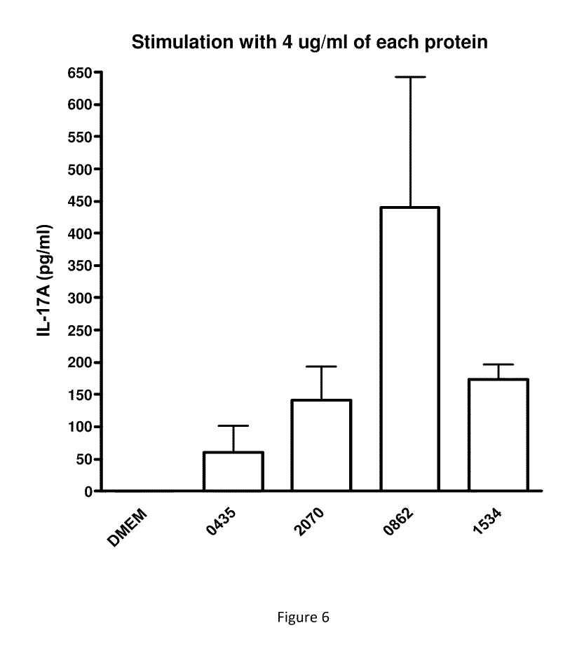

In the next immunization study, the three proteins contained within the combination were used individually as vaccines (FIG. 6). Another protein (SPN 0862), which elicited IL-17A in the splenocyte stimulation assay, was also tested. A group of animals immunized with a protein that had not been stimulatory in any of the cellular assays (SPN 2092) were included as a negative control. All proteins were further purified by gel filtration to remove any contaminants prior to use as individual vaccines.

We claim:

1. A method for obtaining an immunogen or immunogens from a pathogen, comprising the steps of:
killing a pathogen culture with an organic solvent;
removing the solvent;
resuspending the killed bacteria in aqueous solution;
removing particulates from the aqueous solution;
whereby immunogens are retained in the aqueous solution.

2. A method of claim 1, where the pathogen is a bacteria, virus, fungi, or parasite.

3. A method of claim 1, where the immunogen is a protein, carbohydrate, lipid, nucleic acid, or small molecule derived from the pathogen

4. The method of claim 1, further comprising the steps of:
Isolating the proteins within the aqueous solution;
Determining specific antibody or T-cell activity of the isolated antigens, in combination or singly

5. A method for obtaining bacterial T-cell-stimulating immunogens comprising the steps of:
killing a bacterial culture with an organic solvent;
removing the solvent;
resuspending the killed bacteria in aqueous solution;
removing particulates from the aqueous solution;
whereby T-cell immunogens are retained in the aqueous solution.

6. The method of claim 5, further comprising the steps of:
isolating the proteins within the aqueous solution;
determining the Th17-cell inducing activity of the isolated proteins, in combination or singly.

7. The method of claim 3, wherein said culture is a culture of Streptococcus pneumoniae.

8. The method of claim 5, wherein said organic solvent is choloroform.

9. The method of claim 1, wherein said immunogen is further prepared as a vaccine that reduces or protects a mammal against pneumococcal colonization.

10. The method of claim 9, wherein said vaccine further comprises an adjuvant.

11. The method of claim 9, wherein said vaccine is administered mucosally.

12. The method of claim 1, wherein said immunogen is at least one of the pneumococcal proteins SP0862, SP1534, and SP2070.

13. A pharmaceutical composition for eliciting an immune response in a mammal comprising pneumococcal proteins SP0862, SP1534, and SP2070.

14. A pharmaceutical composition for eliciting an immune response in a mammal comprising the T-cell stimulating immunogens prepared by the method of claim 1.
